(19) United States
(12) Patent Application Publication
(10) Pub. No.: US 2003/0167051 A1
(43) Pub. Date: Sep. 4, 2003

(54) Intravascular catheter shaft

(76) Inventor: Pu Zhou, Maple Grove, MN (US)

Correspondence Address: CROMPTON, SEAGER & TUFTE, LLC, 1221 NICOLLET AVENUE, SUITE 800, MINNEAPOLIS, MN, 55403-2420, US

(21) Appl. No.: 10/377,457

(22) Filed: Feb. 28, 2003

Related U.S. Application Data
(60) Provisional application No. 60/361,229, filed on Feb. 28, 2002.

(51) Int. Cl. A61M 25/00

(52) U.S. Cl. 604/523; 138/137; 428/35.9

(57) ABSTRACT

A catheter shaft includes a polyoxymethylene-polyurethane elongate shaft including a proximal portion having about 80 to about 95 weight % polyoxymethylene and about 5 to about 20 weight % polyurethane, an intermediate portion having about 20 to about 50 weight % polyoxymethylene and about 50 to about 80 weight % polyurethane, and a distal portion having about 5 to about 20 weight % polyoxymethylene and about 80 to about 95 weight % polyurethane. The intermediate portion is disposed between the proximal portion and the distal portion.

### RELATED APPLICATION

This application claims the benefit of priority under 35 U.S.C § 119(e) to U.S. Provisional Application Serial No. 60/361,229, filed Feb. 28, 2002, which is herein incorporated by reference.

### FIELD OF THE INVENTION

The present invention generally relates to intravascular catheter shafts. More specifically, the present invention relates to intravascular catheter shafts for guide catheters, diagnostic catheters, balloon catheters, and the like.

### BACKGROUND OF THE INVENTION

Diagnostic catheters and guide catheters are commonly used to facilitate the diagnosis and treatment of vascular diseases such as coronary artery disease and peripheral vascular disease. Balloon catheters are commonly used to treat vascular disease by dilating stenotic lesions. Because such intravascular catheters must be navigated to remote vascular sites through vascular anatomy that may be very tortuous, it is desirable to have a catheter shaft that exhibits improved torqueability, trackability and pushability.

### SUMMARY OF THE INVENTION

The invention provides several alternative designs, materials and methods of manufacturing alternative catheter structures and assemblies.

One embodiment includes a catheter shaft having a polyoxymethylene-polyurethane elongate shaft including a proximal portion having about 80 to about 95 weight % polyoxymethylene and about 5 to about 20 weight % polyurethane, an intermediate portion having about 20 to about 50 weight % polyoxymethylene and about 50 to about 80 weight % polyurethane, and a distal portion having about 5 to about 20 weight % polyoxymethylene and about 80 to about 95 weight % polyurethane. In one alternative embodiment, the distal portion has about 0 to about 5 weight % polyoxymethylene and about 95 to about 100 weight % polyurethane. The intermediate portion is disposed between the proximal portion and the distal portion.

A further embodiment includes a catheter shaft having a polyoxymethylene-polyurethane elongate shaft including a proximal portion having a flexural modulus of about 380 to about 210 ksi, an intermediate portion having a flexural modulus of about 30 to about 90 ksi, and a distal portion having a flexural modulus of less than about 30 ksi. The intermediate portion is disposed between the proximal portion and the distal portion.

A further embodiment includes a catheter having an inner tubular member having a proximal end, a distal end and a longitudinal surface. A support member is disposed over a substantial portion of the longitudinal surface. The support member has a distal end terminated proximal of the inner tubular member distal end. A polyoxymethylene-polyurethane elongate shaft is disposed over the inner tubular member. The polyoxymethylene-polyurethane elongate shaft includes a proximal portion having about 80 to about 95 weight % polyoxymethylene and about 5 to about 20 weight % polyurethane, an intermediate portion having about 20 to about 50 weight % polyoxymethylene and about 50 to about 80 weight % polyurethane, and a distal portion having about 5 to about 20 weight % polyoxymethylene and about 80 to about 95 weight % polyurethane. In one alternative embodiment, the distal portion has about 0 to about 5 weight % polyoxymethylene and about 95 to about 100 weight % polyurethane. The intermediate portion is disposed between the proximal portion and the distal portion.

A further embodiment includes a balloon catheter having an inner tubular member having a proximal end, a distal end and a longitudinal surface. A support member is disposed over a substantial portion of the longitudinal surface. The support member has a distal end terminated proximal of the inner tubular member distal end. A polyoxymethylene-polyurethane elongate shaft includes a proximal portion having about 80 to about 95 weight % polyoxymethylene and about 5 to about 20 weight % polyurethane, an intermediate portion having about 20 to about 50 weight % polyoxymethylene and about 50 to about 80 weight % polyurethane, and a distal portion having about 5 to about 20 weight % polyoxymethylene and about 80 to about 95 weight % polyurethane. In one alternative embodiment, the distal portion has about 0 to about 5 weight % polyoxymethylene and about 95 to about 100 weight % polyurethane. The intermediate portion is disposed between the proximal portion and the distal portion. A balloon is disposed about the distal portion of the polyoxymethylene-polyurethane elongate shaft.

The above summary of some embodiments is not intended to describe each disclosed embodiment or every implementation of the present invention. The Figures, and Detailed Description which follow more particularly exemplify these embodiments.

### BRIEF DESCRIPTION OF THE DRAWINGS

The invention may be more completely understood in consideration of the following detailed description of various embodiments of the invention in connection with the accompanying drawings in which:

FIG. 1 is a plan view of an intravascular catheter in accordance with an embodiment of the present invention shown as a guide or diagnostic catheter;

FIG. 2 is a cross-sectional view taken along line 2-2 in FIG. 1;

FIG. 3 is a longitudinal sectional view taken along line 3-3 in FIG. 1;

FIG. 4 is a plan view of an intravascular catheter in accordance with another embodiment of the present invention shown as a balloon catheter; and

FIG. 5 is a cross-sectional view taken along line 5-5 in FIG. 4.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION OF THE INVENTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

Weight percent, percent by weight, wt %, wt-%, % by weight, and the like are synonyms that refer to the concentration of a substance as the weight of that substance divided by the weight of the composition and multiplied by 100.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention.

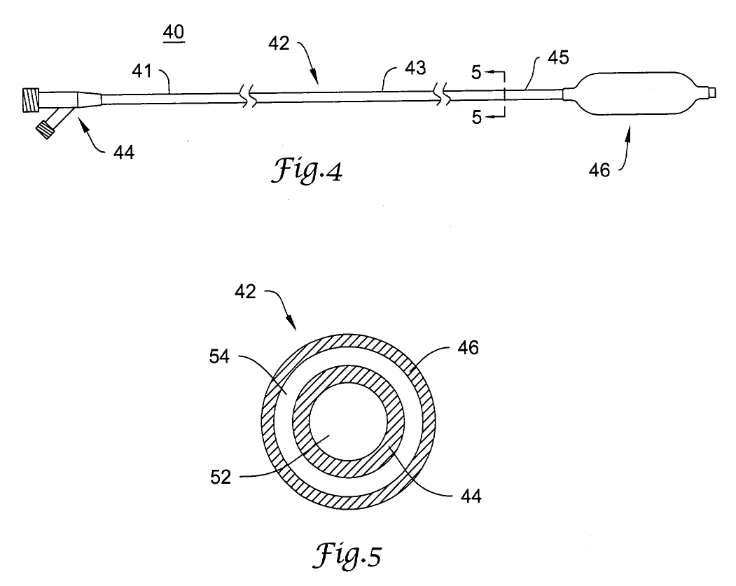

Refer now to FIG. 1 which illustrates an intravascular catheter in accordance with an embodiment of the present invention. For purposes of illustration and discussion only, the intravascular catheter shown in FIG. 1 is in the form of a guide or diagnostic catheter 10, but may comprise virtually any catheter used for intravascular applications. For example, the intravascular catheter may comprise a balloon catheter 40 as discussed with reference to FIG. 4.

The guide or diagnostic catheter 10 may have a length and an outside diameter sufficient to enable intravascular insertion and navigation. For example, the catheter 10 may have a length of approximately 100 cm to 150 cm and an outside diameter of approximately 4 to 9 French. Guide or diagnostic catheter 10 may be substantially conventional except as described herein and shown in the drawings.

The catheter 10 includes an elongate shaft 12 having a proximal portion 17, a distal portion 19 and an intermediate portion 15 disposed between the proximal portion 17 and the distal portion 19. A distal tip 16 is connected to the distal end of the elongate shaft 12. The distal tip 16, the distal portion 19 and the intermediate portion 15 of the elongate shaft 12 may be curved depending on the particular clinical application. The elongate shaft 12 and the distal tip 16 include a lumen 18 (shown in FIG. 4) extending therethrough to facilitate insertion of other medical devices (e.g., guide wires, balloon catheters, etc.) therethrough, and/or to facilitate injection of fluids (e.g., radiopaque dye, saline, drugs, etc.) therethrough. A manifold 14 can be connected to the proximal end of the proximal portion 17 of the elongate shaft 12 to facilitate connection to other medical devices (e.g., syringe, Y-adapter, etc.) and to provide access to the lumen 18.

The proximal portion 17 of the elongate shaft 12 may be from about 60 to about 135 cm or about 60 to about 90% of the total length. The intermediate portion 15 of the elongate shaft 12 may be from about 15 to about 30 cm or about 15 to about 20% of the total length. The distal portion 19 of the elongate shaft 12 may be from about 2 to about 10 cm or about 2 to about 7% of the total length.

The elongate shaft 12 can be formed from a polymer blend of polyoxymethylene and polyurethane hereinafter denoted as “polyoxymethylene-polyurethane”. The proximal portion 17, intermediate portion 15 and distal portion 19 are each formed with a varying amount of polyoxymethylene. The proximal portion 17 can have about 80 to about 95 weight % polyoxymethylene. The intermediate portion 15 can have about 20 to about 50 weight % polyoxymethylene. The distal portion 19 can have about 5 to about 20 weight % polyoxymethylene. In an alternative embodiment, the distal portion can include about 0 to about 5 weight % polyoxymethylene.

The proximal portion 17, intermediate portion 15 and distal portion 19 are each formed with a varying amount of polyurethane. The proximal portion 17 can have about 5 to about 20 weight % polyurethane. The intermediate portion 15 can have about 50 to about 80 weight % polyurethane. The distal portion 19 can have about 80 to about 95 weight % polyurethane. In one alternative embodiment, the distal portion can include about 95 to about 100 weight % polyurethane.

The proximal portion 17, intermediate portion 15 and distal portion 19 are each formed with varying flexural modulus. The proximal portion 17 can have a flexural modulus of about 210 to about 380 ksi. The intermediate portion 15 can have a flexural modulus of about 30 to about 90 ksi. The distal portion 19 can have a flexural modulus of less than about 30 ksi or from about 1 to about 30 ksi or from about 15 to about 30 ksi.

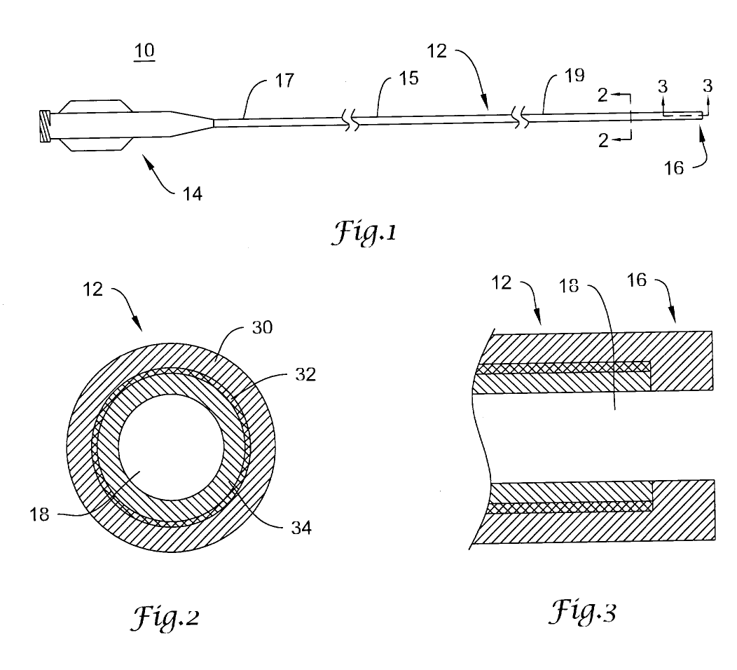

As best seen in FIGS. 2 and 3, the elongate shaft 12 may be multi-layered (shown) or uni-layered (not shown). In the multi-layer embodiment (shown), the elongate shaft 12 may include a polyoxymethylene-polyurethane outer layer 30, a reinforcement layer 32 and an inner layer 34. In the uni-layer embodiment (not shown), the elongate shaft 12 may comprise a single polyoxymethylene-polyurethane layer 30. The distal tip 16 may comprise the outer layer 30 extending beyond the inner layer 34 and the reinforcement layer 32 to define a soft atraumatic tip.

The inner layer 34 may comprise a lubricious polymer such as high-density polyethylene (HDPE) or polytetrafluoroethylene (PTFE), for example. Alternatively, the inner layer 34 may comprise a polyoxymethylene homopolymer or a polyoxymethylene blend as discussed herein with reference to the outer layer 30. The reinforcement layer 32 may comprise a braided metallic wire or coil, for example.

The outer layer 30 may be formed, for example, by extrusion of a polyoxymethylene pre-blend or by co-extrusion of the polyoxymethylene with the polyurethane such as by interrupted layer co-extrusion (ILC). Alternatively, the outer layer 30 may be formed of separated extruded tubular segments subsequently fused together. A suitable polyoxymethylene is commercially available under the trade name Delrin™ commercially available from DuPont Wilmington, Del.

FIG. 4 illustrates an intravascular catheter in accordance with another embodiment of the present invention. For purposes of illustration and discussion only, the intravascular catheter shown in FIG. 4 is in the form of an intravascular balloon catheter 40. The balloon catheter 40 may be substantially conventional except as described herein and shown in the drawings.

The balloon catheter 40 includes an elongate shaft 42 having a proximal portion 41, a distal portion 45 and an intermediate portion 43 disposed between the proximal portion 41 and the distal portion 45. An inflatable balloon 46 is connected to the distal portion 45 of the elongate shaft 42. Depending on the type (over-the-wire, fixed-wire, single-operator-exchange, etc.) of balloon catheter 40, all or a portion of the elongate shaft 42 may include an inner tube 44 defining a guide wire lumen 52 therein, and an outer tube 46 disposed thereon to define an annular inflation lumen 54 therebetween. A manifold 44 can be connected to the proximal end of the proximal portion 41 of the elongate shaft 42 to facilitate connection to other medical devices (e.g., syringe, Y-adapter, etc.) and to provide access to the lumen 18.

The inner tube 44 may comprise a lubricious polymer such as HDPE or PTFE, for example. The outer tube 46 may comprise a polymer blend that is similar to or the same as the polymer blends of the outer layer 30 discussed with reference to FIG. 1. In addition, the manufacture and arrangement of parts for outer tube 46 may be similar to or the same as that discussed with reference to the outer layer 30.

### EXAMPLES

The following polyoxymethylene blend material test data is provided by way of example, not limitation:

| Sample No. | Composition |
|---|---|
| 1 | 100% Polyoxymethylene—Delrin 150SA |
| 2 | 20% Urethane |
| | 80% Polyoxymethylene |
| 3 | 40% Urethane |
| | 60% Polyoxymethylene |
| 4 | 50% Urethane |
| | 50% Polyoxymethylene |
| 5 | 60% Urethane |
| | 40% Polyoxymethylene |

| Test | Sample 1 | Sample 2 | Sample 3 | Sample 4 | Sample 5 |
|---|---|---|---|---|---|
| Tensile St at | 9830 | 7020 | 5170 | 4360 | 3090 |
| Yield (psi) | | | | | |
| Elongation at | 19 | 27 | 42 | 50 | 150 |
| Yield (%) | | | | | |
| Tensile St at | 9670 | 5660 | 5270 | 4600 | 7260 |
| Break (psi) | | | | | |
| Elongation at | 37 | 100 | 400 | 330 | 480 |
| Break (%) | | | | | |
| Tensile Set | 6 | 49 | 310 | 240 | 310 |
| at Break (%) | | | | | |
| Tensile | 412600 | 237600 | 149400 | 115800 | 30530 |
| Modulus | | | | | |
| (psi) | | | | | |
| Flexural | 397200 | 218100 | 117800 | 92670 | 46930 |
| Modulus | | | | | |
| (psi) | | | | | |
| Shore D | 80 | 75 | 65 | 58 | 50 |
| Hardness | | | | | |
| (points) | | | | | |
| Vicat | 173.7 | 171.9 | 166.2 | 150.8 | 134.3 |
| Softening | | | | | |
| Point (° C.) | | | | | |
| DSD Melt | 189 | 189.8 | 188.6 | 183.8 | 183.7 |
| Point (° C.) | | | | | |
| Melt Index | 10.9 | 8.7 | 8 | 6.4 | 6.2 |

The polyoxymethylene-polyurethane blends of the present invention have been found to provide catheter shafts with enhanced stiffness, kink resistance, and curve performance. For example, kink test results indicate that catheter tubes formed of polyoxymethylene-polyurethane blends are more kink resistant than the prior art.

The present invention should not be considered limited to the particular examples described above, but rather should be understood to cover all aspects of the invention as fairly set out in the attached claims. Various modifications, equivalent processes, as well as numerous structures to which the present invention may be applicable will be readily apparent to those of skill in the art to which the present invention is directed upon review of the instant specification. It should be understood that this disclosure is, in many respects, only illustrative. Changes may be made in details, particularly in matters of shape, size, and arrangement of steps without exceeding the scope of the invention. The scope of the invention is, of course, defined in the language in which the appended claims are expressed.

What is claimed is:

1. A catheter shaft comprising:
a polyoxymethylene-polyurethane elongate shaft including a proximal portion having about 80 to about 95 weight % polyoxymethylene and about 5 to about 20 weight % polyurethane, an intermediate portion having about 20 to about 50 weight % polyoxymethylene and about 50 to about 80 weight % polyurethane, and a distal portion having about 5 to about 20 weight % polyoxymethylene and about 80 to about 95 weight % polyurethane, wherein the intermediate portion is disposed between the proximal portion and the distal portion.

2. The catheter shaft according to claim 1, further comprising an inner polytetrafluoroethylene tubular member disposed within the polyoxymethylene-polyurethane elongate shaft.

3. The catheter shaft according to claim 2, further comprising a braided metallic support member disposed between the inner polytetrafluoroethylene tubular member and the polyoxymethylene-polyurethane elongate shaft.

4. The catheter shaft according to claim 1, wherein the proximal portion, intermediate portion and distal portion define a total shaft length and the proximal portion is about 60 to about 90% of the total length, the intermediate portion is about 15 to about 20% of the total length, and the distal portion is about 2 to about 7% of the total length.

5. A catheter shaft comprising:
a polyoxymethylene-polyurethane elongate shaft including a proximal portion having a flexural modulus of about 380 to about 210 ksi, an intermediate portion having a flexural modulus of about 30 to about 90 ksi, and a distal portion having a flexural modulus of less than about 30 ksi, wherein the intermediate portion is disposed between the proximal portion and the distal portion.

6. The catheter shaft according to claim 5, further comprising an inner polytetrafluoroethylene tubular member disposed within the polyoxymethylene-polyurethane elongate shaft.

7. The catheter shaft according to claim 6, further comprising a braided metallic support member disposed between the inner polytetrafluoroethylene tubular member and the polyoxymethylene-polyurethane elongate shaft.

8. The catheter shaft according to claim 6, wherein the proximal portion, intermediate portion and distal portion define a total shaft length and the proximal portion is about 60 to about 90% of the total length, the intermediate portion is about 15 to about 20% of the total length, and the distal portion is about 2 to about 7% of the total length.

9. A catheter comprising:
an inner tubular member having a proximal end, a distal end and a longitudinal surface;
a support member disposed over a substantial portion of the longitudinal surface, the support member having a distal end terminated proximal of the inner tubular member distal end; and
a polyoxymethylene-polyurethane elongate shaft disposed over the inner tubular member, the polyoxymethylene-polyurethane elongate shaft including a proximal portion having about 80 to about 95 weight % polyoxymethylene and about 5 to about 20 weight % polyurethane, an intermediate portion having about 20 to about 50 weight % polyoxymethylene and about 50 to about 80 weight % polyurethane, and a distal portion having about 5 to about 20 weight % polyoxymethylene and about 80 to about 95 weight % polyurethane, wherein the intermediate portion is disposed between the proximal portion and the distal portion.

10. The catheter according to claim 9, wherein the inner tubular member comprises polytetrafluoroethylene.

11. The catheter according to claim 9, wherein the support member comprises a braided metallic member.

12. The catheter according to claim 9, wherein a manifold is disposed at the proximal end of the inner tubular member.

13. The catheter according to claim 9, wherein the proximal portion, intermediate portion and distal portion define a total shaft length and the proximal portion is about 60 to about 90% of the total length, the intermediate portion is about 15 to about 20% of the total length, and the distal portion is about 2 to about 7% of the total length.

14. A balloon catheter comprising:
an inner tubular member having a proximal end, a distal end and a longitudinal surface;
a support member disposed over a substantial portion of the longitudinal surface, the support member having a distal end terminated proximal of the inner tubular member distal end;
a polyoxymethylene-polyurethane elongate shaft including a proximal portion having about 80 to about 95 weight % polyoxymethylene and about 5 to about 20 weight % polyurethane, an intermediate portion having about 20 to about 50 weight % polyoxymethylene and about 50 to about 80 weight % polyurethane, and a distal portion having about 5 to about 20 weight % polyoxymethylene and about 80 to about 95 weight % polyurethane, wherein the intermediate portion is disposed between the proximal portion and the distal portion; and
a balloon disposed about the distal portion of the polyoxymethylene-polyurethane elongate shaft.

15. The balloon catheter according to claim 14, wherein the inner tubular member comprises polytetrafluoroethylene.

16. The balloon catheter according to claim 14, wherein the support member comprises a braided metallic member.

17. The balloon catheter according to claim 14, further comprising a manifold disposed at the proximal end of the inner tubular member.

18. The balloon catheter according to claim 14, wherein the inner tubular member defines a guidewire lumen and an inflation lumen is defined between the inner tubular member and the polyoxymethylene-polyurethane elongate shaft.

19. The balloon catheter according to claim 14, wherein the proximal portion, intermediate portion and distal portion define a total shaft length and the proximal portion is about 60 to about 90% of the total length, the intermediate portion is about 15 to about 20% of the total length, and the distal portion is about 2 to about 7% of the total length.
